(19) United States
(12) Patent Application Publication
(10) Pub. No.: US 2016/0324904 A1
(43) Pub. Date: Nov. 10, 2016

(54) SUPPLEMENT FOR IMPROVING IMMUNOTOLERANCE, INTESTINAL ENVIRONMENT AND INTESTINAL TRACT BARRIER

(71) Applicant: Ryozo SAITO, Tokyo (JP)

(72) Inventor: Ryozo SAITO, Tokyo (JP)

(21) Appl. No.: 14/704,483

(22) Filed: May 5, 2015

(51) Int. Cl. A61K 35/744; A61K 31/07; A61K 31/59; A61K 38/16; A61K 31/315; A61K 33/06; A61K 33/08; A61K 31/198; A61K 31/715

(52) U.S. Cl. CPC A61K 35/744; A61K 31/198; A61K 31/07; A61K 31/59; A61K 31/715; A61K 31/315; A61K 33/06; A61K 33/08; A61K 38/168

(57) ABSTRACT

A supplement for improving immunotolerance, intestinal environment and intestinal tract barrier for the purpose of improving intestinal vulnerability, food intolerance or allergy, systemic allergy, autoimmune diseases and systemic inflammatory diseases by immune reactions which is obtained by mixing an ingredient (A) comprising at least one of a probiotic that is any one of lactic acid bacteria, Lactobacillus bifidus, saccharified bacteria, natto bacteria and yeast fungus, or a mixture thereof, a biogenic that is a bacteria-producing substance such as lactic acid bacteria, and a prebiotic is indigestible food ingredients such as oligosaccharide, dietary fiber, substance stimulating production of Lactobacillus bifidus, and others or a mixture thereof and an ingredient (B) comprising at least one of L-glutamine, zinc, vitamin A and vitamin D.

### TECHNICAL FIELD

The present invention relates to a supplement for improving immunotolerance, intestinal environment and intestinal tract barrier for the purpose of improving intestinal vulnerability, food intolerance or allergy, systemic allergy, autoimmune diseases and systemic inflammatory diseases by immune reactions.

### BACKGROUND ART

A nutritional supplement food and a health food are suggested in, for example, Japanese Patent Application Laid-Open (JP-A) No. 2009-159873, which is characterized in that the nutritional supplement food and the health food containing one or two or more ingredients selected from vitamin, mineral, amino acid, peptide, protein, saccharide, oligosaccharide, polysaccharide, lipid, nucleic acid, and other food materials having health maintenance and promotion action and the like for supplying the ingredients are added at the time of eating an existing food whereby to be eaten simultaneously with the food.

In the nutritional supplement food and the health food pertaining to the invention described in the above-described Patent Document 1, compatibility with an existing food that is the subject of the addition was considered at the time of the product design, but improvement of intestinal vulnerability and intestinal tract allergy was not aimed at the time of the product design.

### SUMMARY OF INVENTION

### Technical Problem

The problem of this invention is to provide a supplement for improving immunotolerance, intestinal environment and intestinal tract barrier, which decreases the intestinal environment/barrier index, and which is obtained by blending food materials having health maintenance and promotion action so as to improving intestinal vulnerability, food intolerance or allergy, systemic allergy, autoimmune diseases and systemic inflammatory diseases by immune reactions.

To solve the above-described problem, the invention provides a supplement for improving immunotolerance, intestinal environment and intestinal tract barrier, which decreases the intestinal environment/barrier index (described later), and which is obtained by mixing an ingredient (A) and an ingredient (B),
the ingredient (A) comprise, at least any one of a probiotic, a biogenic and a prebiotic,
the probiotic is a microorganism giving beneficial effects to the host by improving balance of intestinal microorganism flora,
the biogenic is a food ingredient directly acting on a living body without intervention of intestinal flora,
the prebiotic is a substance selectively acting on useful bacteria living in the intestine, and promoting proliferation thereof or elevating activity thereof whereby to advantageously act on the health of the host, and
the ingredient (B) comprising at least one of L-glutamine, zinc, vitamin A (hereinafter, V. A) and vitamin D (hereinafter, V. D),
the probiotic corresponds to any one of lactic acid bacteria, Lactobacillus bifidus, saccharified bacteria, natto bacteria, yeast fungus spore lactic acid bacteria, and others, or a mixture thereof, the biogenic corresponds to any one of casein phosphopeptide of a bioactive peptide, lactoferrin of multifunctional whey protein and docosahexaenoic acid or eicosapentaenoic acid that is one kind of polyunsaturated fatty acid, or a mixture thereof, and the prebiotic corresponds to indigestible food ingredients such as oligosaccharide, dietary fiber, substance stimulating production of Lactobacillus bifidus, and others or a mixture thereof.

According to the supplement for improving immunotolerance, intestinal environment and intestinal tract barrier pertaining to the invention, this invention makes it possible to improve intestinal vulnerability, food intolerance or allergy, systemic allergy, autoimmune diseases and systemic inflammatory diseases by immune reactions by improving the human intestinal environment and the intestinal tract barrier, decreasing development of inflammation caused by antigen-antibody reaction in the intestinal tract and/or in the body system, increasing suppression of destructive fungus, production of nutrition, promotion of intestinal tract peristalsis and activation of mucous membrane defense function, which are originally performed by intestinal bacteria, and decreasing (normalizing) the intestinal environment/barrier index.

### BRIEF DESCRIPTION OF DRAWINGS

FIG. 1 is a diagram showing the intestinal environment/barrier index (Ig G score) for dairy products, seafood, meats and eggs in comparison before and after intake of the supplement pertaining to Examples in the first subject.

FIG. 2 is a diagram showing the intestinal environment/barrier index for similar vegetables, nuts and cereals and other foods in comparison.

FIG. 3 is a diagram showing the intestinal environment/barrier index for vegetables, nuts and cereals and other foods in comparison before and after intake of the supplement pertaining to Examples in the second subject.

FIG. 4 is a diagram showing the intestinal environment/barrier index for similar dairy products, seafood, meats and eggs, fruits and spices in comparison.

### DESCRIPTION OF EMBODIMENTS

Hereinafter, embodiments of the invention will be described in detail.

The supplement for improving immunotolerance, intestinal environment and intestinal tract barrier pertaining to the invention is formed by mixing the ingredient (A) comprising at least one of a probiotic, a biogenic and a prebiotic, and the ingredient (B) comprising at least one of L-glutamine, zinc, V. A and V. D.

The “probiotic” is a “microorganism giving beneficial effects to a host animal by improving balance of intestinal microorganism flora”, and corresponds to lactic acid bacteria, Lactobacillus bifidus, natto bacteria, butyric acid bacteria spore lactic acid bacteria, and the like; the “prebiotic” is a “substance selectively acting on useful bacteria living in the intestine, and promoting proliferation thereof or elevating activity thereof whereby to advantageously act on the health of a host”, and corresponds to indigestible food ingredients such as oligosaccharide, dietary fiber (polysaccharides) and B.G.S (substance stimulating production of Lactobacillus bifidus); and the “biogenic” is a “food ingredient directly acting on regulation of a living body, defense of a living body, disease prevention, recovery, aging control and the like without intervention of intestinal flora, such as CPP (casein phosphopeptide) of a bioactive peptide, LPS (lipopolysaccharide), lactoferrin of multifunctional whey protein and DHA (docosahexaenoic acid) or EPA (eicosapentaenoic acid)that is one kind of polyunsaturated fatty acid.

The origosaccharide is including, garactorigosaccharide (GOS), fructooligosaccharide (FOS), mannan oligosaccharide-based nutritional supplements (MOS), arabinogalactan, cyclodextrin and lactulose. The dietary fiber is including guar gum (polysaccharides including 2 molecules of mannose in normal chain combination with one molecule of galactose in side chain combination), digestion resistant dextrin, pectin, inulin.

In this embodiment, at least one of a probiotic, a biogenic and a prebiotic increases the number of beneficial bacteria-called intestinal bacteria, and decreases bad bacteria-called intestinal bacteria to decrease the intestinal environment/barrier index, whereby to promote suppression of destructive fungus, production of nutrition, promotion of intestinal tract peristalsis and activation for mucous membrane defense function, which are originally performed by intestinal bacteria and improve intestinal environment.

Specifically, the suppression of destructive fungus results from inhibition to fixation of destructive fungus, lowering of pH by an organic acid (lactic acid, acetic acid), and production of an antibacterial substance (bacteriocin). In addition, the production of nutrition refers to decomposition of indigestible polysaccharides, vitamin production (V. B₅, V. H, V. K), and production of short-chain fatty acids (butyric acid) that become nutrients of the epithelium of the intestinal tract. The activation for mucous membrane defense function refers to enhancement of production of secretory IgA and type C secretin.

Immunoglobulin G (Ig G) makes up 70% or more of the antibodies of the blood plasma (IgE is present only in 0.001% or less.), and plays a role of neutralizing the antibodies, and presenting an antigen in cell-mediated immunity. The cell-mediated immunity is called type IV allergy (delayed type), and expresses allergy reaction over several hours to several days.

Ig G passes the placenta, and helps the immunity of a newborn baby after birth. In addition, the retention time in the body is about 3 weeks.

When a cause for food intolerance or allergy is considered, a food-derived protein is usually not present in the blood, and thus antibody production cannot be present. In other words, food antigen-specific Ig G is normally not present. However, undigested protein is absorbed by vulnerability of the intestinal tract barrier from disorder of the intestinal environment and the like, and indigestion, and thus Ig G against the food protein is produced, and as a result, food antigen-specific Ig G is detected in the blood.

The intestinal environment/barrier index is a scored value of Ig G specific to a food antigen inducing delayed food allergy, and “0” indicating no detection is normal, and as the index is higher, disorder of any one of the intestinal environment and the intestinal tract barrier is presumed.

In addition, with concomitant use of the ingredient (B) described below with the ingredient (A) comprising at least one of a probiotic, a biogenic and a prebiotic as described above, the invention recovers the intestinal environment and the barrier function of the intestinal tract whereby to improve allergy against a food antigen, particularly delayed type food allergy induced by Ig G.

At least one of a probiotic, a biogenic and a prebiotic described above may be used as the ingredient (A), and the ingredient (B) that is concomitantly used with the ingredient (A) comprises at least one (one or two or more) of L-glutamine, zinc, V. A and V. D.

Further, the ingredient (A) may comprise of L-glutamine and the ingredient (B) may comprise at least one of Zinc, Vitamin A and Vitamin D.

Herein, the zinc may include at least zinc among zinc and zinc gluconate, and the V. A may include at least V. A among V. A, pre-V. A, carotenoid, and derivatives of V. A, and the V. D may include at least V. D among V. D, pre-V. D and derivatives of V. D.

Furthermore, the following ingredient (C) may be added to and mixed with the ingredient (A) and the ingredient (B). The ingredient (C) comprises at least one of V. B group; calcium; magnesium; a magnesium compound; a functional nutritional ingredient comprising at least one of quercetin, lactoferrin, LPS (lipopolysaccharide) and curcumin; protein; and amino acid.

Specifically, spore lactic acid bacteria may be used as the probiotic. In addition, the prebiotic may include at least enzymatic-decomposition products of guar gum that is a water-soluble dietary fiber among enzymatic-decomposition products of guar gum, isomal oligosaccharide, fructo-oligosaccharide, arabinogalactan, lactulose and cyclic oligosaccharide.

Furthermore, in the ingredient (B), the zinc may include at least zinc among zinc and zinc gluconate, the V. A may include at least V. A among V. A, pre-V. A, carotenoid and derivatives of V. A, and the V. D may include at least V. D among V. D, pre-V. D and derivatives of V. D.

In addition, as the ingredient (C), a protein may be used in addition to calcium; magnesium; a magnesium compound and the like described above. The calcium may include shell calcium, the magnesium compound may include magnesium oxide, the protein may include soybean peptide having low antigenicity, and the amino acid may include L methionine for improving the amino acid score of soybean peptide.

Further, the ingredient (A) may comprise of L-glutamine and the ingredient (B) may comprise at least one of Zinc, Vitamin A and Vitamin D.

With concomitant use of at least one of zinc or a zinc compound, V. A (particularly V. A), or V. D (particularly V. D) as the ingredient (B) with the probiotic and/or prebiotic, they may perform effective regeneration, restoration of the epithelium cell of the intestinal tract and may induct immunotolerance because they are essential nutrients when the epithelium cell of the intestinal tract replaces the old with the new performing cell division. In addition, with concomitant use of the biogenic and the ingredient (B), regulation of the function of the intestine, suppression of rotting in the intestine and induction of immunotolerance can be performed.

Particularly, V. A and V. D act on an antigen presentation cell that is in the vicinity of the epithelium of the intestinal tract or in other lymphoid tissues and carries immunity response; a helper T cell; a B cell performing antibody production, and are essential nutrients for optimal immunity response, and thus suppress induction of disadvantageous immune response such as allergy and self-antibody production from deficiency thereof.

With concomitant use of the ingredient (C), that is, V. B group, calcium, magnesium, or a compound thereof or a functional nutritional ingredient such as quercetin, lactoferrin and curcumin with any one of those described above, calcium, magnesium or a compound thereof can control the fecal properties, and quercetin or the like has anti-inflammation action. If protein or amino acid necessary for protein synthesis is added, they can reinforce effective regeneration and restoration of the epithelium cell of the intestinal tract and also add functionality. Folic acid may be added to maintain the function of immunological tolerance induction.

Due to abuse of antibiotics and anti-inflammatory analgesics in recent years, the barrier function of the intestinal tract is vulnerable. In addition, due to overeating, overdrinking, food contamination by residual agricultural chemicals or the like, decrease of the amount of dietary fiber intake and the like, intestinal bacteria are damaged, and thus the epithelium cell of the intestinal tract, which uses fatty acid decomposed by the intestinal bacteria as a nutrition source, becomes vulnerable, and the epithelium cell of the intestinal tract is increasingly damaged by exotoxin produced by bad bacteria increasing.

As a result from those described above, undigested protein reaches antigen presentation cells in the vicinity of the intestinal tract, and immune response with respect to a food protein thereof is induced, whereby to exhibit various symptoms by inflammation caused by local or systemic antigen-antibody reaction.

For example, there are reflux esophagitis, obesity or edema, atopic dermatitis and the like. Any one of them shows specific Ig G positive examples in many cases, and alleviation of the symptoms or fundamental recovery can be obtained by measuring Ig G and combining administration of the supplement of the invention which decreases the intestinal environment/barrier index with removal of highly reactive food.

Next, the amounts of the ingredient (A) and the ingredient (B) contained in recommended single intake amount in an embodiment of the invention will be described. The lower limit value for any one of them represents a number below which the effects cannot be expected. In addition, the upper limit value represents a number as a rule, over which effects do not increase despite over-intake at a time.

Single dose of the ingredient (A) is, for example, such an amount that the number of bacteria is more than 25,000,000 if the bacteria are spore lactic acid bacteria.

The amount of zinc in the ingredient (B) is preferably 10 mg to 120 mg. If the amount of zinc is more than 30 mg, copper shortage may occur due to excessive zinc, and thus copper is added in 5% or more with respect to zinc.

In a case where a main ingredient of the ingredient (B) is L-glutamine, the content of L-glutamine is 100 mg or more. The content of L-glutamine is preferably 1000 mg to 5000 mg. If the content of L-glutamine is more than 1000 mg, absorption of moisture by feces increases due to rapid improvement of colon function, and the feces become hard. As a result, constipation or intestinal blockage may be caused, and thus the seriously ill or the aged need to consider reduction of the use amount, sufficient water taking, and the like. L-glutamine may be an ingredient of the ingredient (B).

Furthermore, when the main ingredient of the ingredient (B) is V. A, pre-V. A, a derivative thereof, 100 IU to 60000 IU is contained as V. A or corresponding amount of carotenoid.

This is because if the content of V. A is less than 100 IU, effective restoration of the epithelium cell of the intestinal tract cannot be achieved, and if the content of V. A is less than the upper limit value, V. A overload is avoided. Meanwhile, when the content of V. A is more than 10000 IU or corresponding amount of carotenoid, the amount is more than the upper limit of recommended intake during pregnancy, and attention should be paid.

When the main ingredient of the ingredient (B) is V. D, pre-V. D or a derivative thereof, 100 IU to 10000 IU is contained as V. D. Administration of V.D may be increased to 100,000 IU at intervals.

If the content of V. D is less than 100 IU, suppression for induction of disadvantageous immune response such as allergy and self-antibody production is insufficient. Meanwhile, if the content of V. D is more than 50000 IU, there may be V. D overload.

### EXAMPLE 1

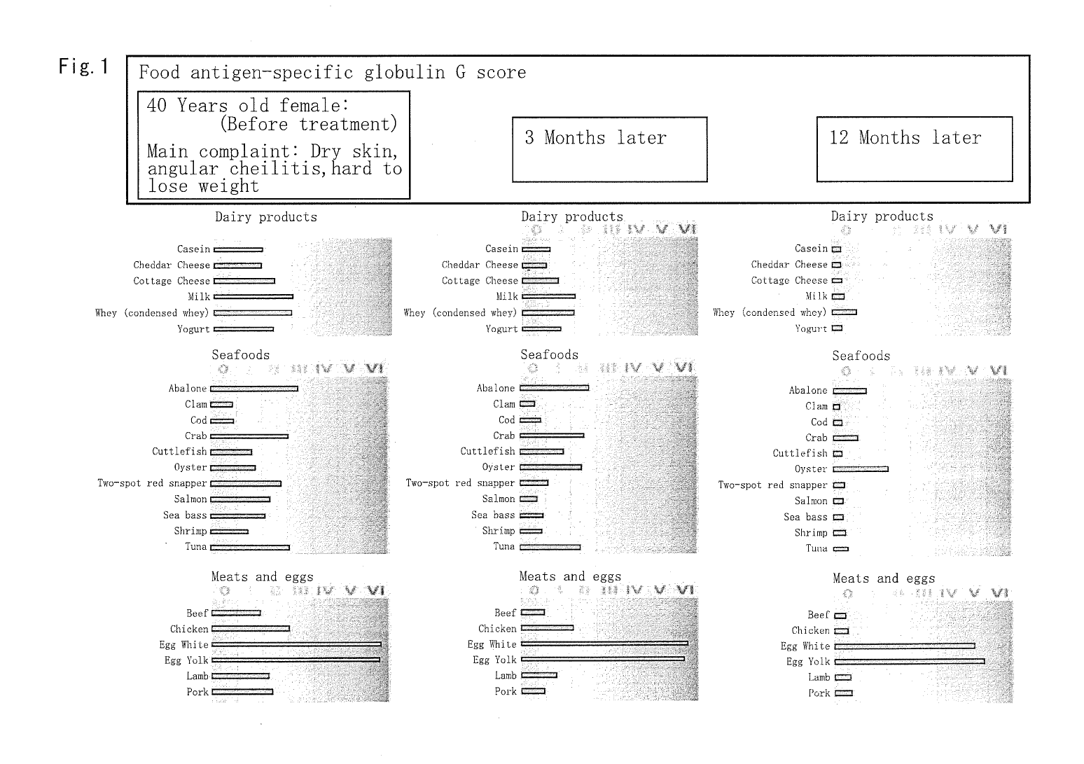

As shown in FIG. 1, the supplement for improving intestinal environment and intestinal tract barrier pertaining to Examples of the invention is constituted by mixing the ingredient (A) containing spore lactic acid bacteria as the probiotic, inulin as the prebiotic, oligosaccharide and cyclic oligosaccharide, the ingredient (B) containing L-glutamine, zinc gluconate, β-carotene, V. A and V. D, vitamin B group containing V. B₁, V. B₂, niacin (V. B₃), V. B₆, V. B₁₂ and folic acid; the ingredient (C) containing shell calcium and pantothenic acid (V. B₅) Ca; Mg oxide; soybean peptide; and L-methionine, and further maltodextrin, which is a polysaccharide, V. C, V. E, biotin and potassium chloride used as a food additive, pectin as a stabilizer, and ferric pyrophosphate, which is iron chloride, and is indicated such that single one packet (30 g) is dissolved in 80 to 180 ml of water, and drunk once to twice per day as a standard.

The ingredients per one packet (30 g) are 14.9 g protein (mainly soybean peptide), 0.4 g lipid, 9.4 g carbohydrate (mainly oligosaccharide, cyclic oligosaccharide and inulin), 192 mg sodium, 1000 IU V. A, 500 IU β-carotene, 10 mg V. B₁, 10 mg V. B₂, 15 mg V. B₆, 100 μg V. B₁₂, 100 mg pantothenic acid Ca, 200 μg folic acid, 100 μg biotin, 30 mg niacin, 100 mg V. C, 800 IU V. D, 20 mg V. E, 150 mg Ca, 150 mg Mg, 0.3 mg Cu, 15 mg Zn, 5 mg Fe and 500 mg Kcl.

### Clinical Example 1

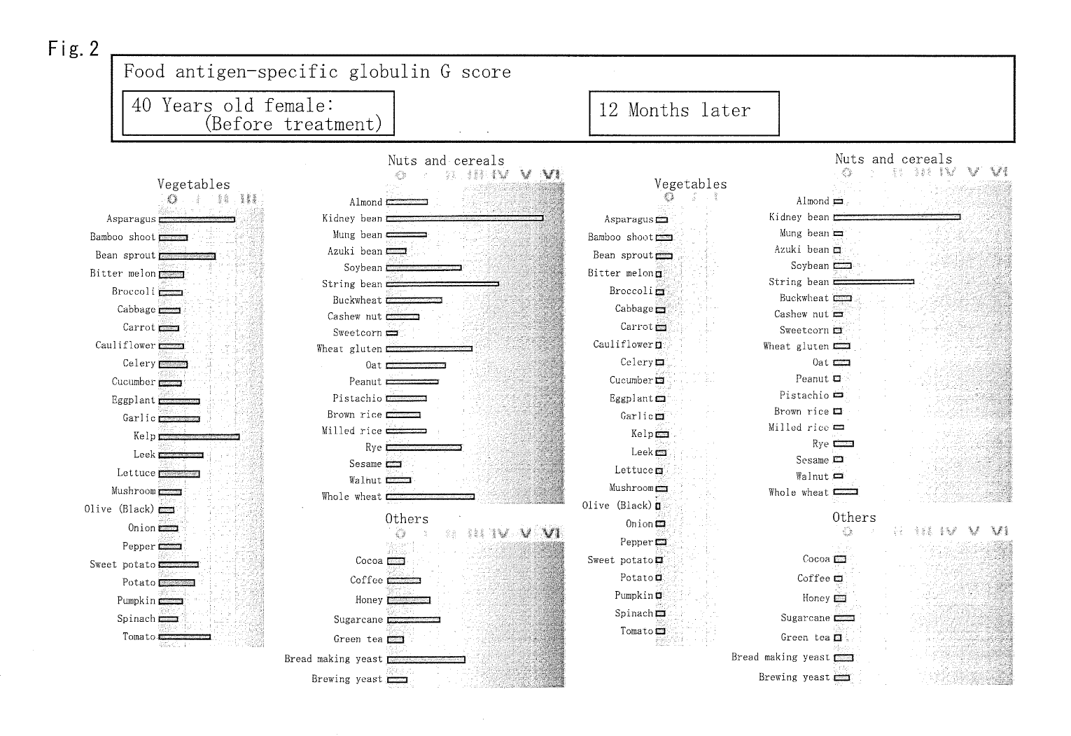

FIGS. 1 and 2 show changes of Ig G scores of each food shown by Japanese standard food panel before the treatment and after initiation of the treatment for 40 years old female in Clinical example 1. Herein, the Ig G score represents severity of non-immediate allergy based on the antibody amount. As to these levels (levels 0 to IV), level 0 represents normal and as the level is higher, it suggests disorder of intestinal environment and intestinal tract barrier as much. In other words, as the level of Ig G score as the intestinal environment/barrier index is smaller, it suggests a state close to normal. Meanwhile, the subject was directed not to intake dairy products and eggs that showed level III or higher at the time of the first medical examination for 12 months.

FIG. 1 shows Ig G scores with respect to dairy products, seafood, meats and eggs before treatment, after 3 months and after 12 months, and FIG. 2 shows Ig G scores with respect to vegetables, nuts and cereals, and others, before treatment and after 12 months, respectively.

In Clinical example 1, as shown in FIG. 1, the Ig G scores for the dairy products were level II or immediately below II, but after 12 months, became level 0 for all. The Ig G score for the seafood decreased slightly for abalone and oyster, but was level 0 for all others. The Ig G score of the meats and the eggs decreased slightly for egg white and egg yolk, but was level 0 for other meats.

In addition, as shown in FIG. 2, the intestinal environment/barrier index greatly improved to level 0 for all of each food of asparagus, bean sprouts, seaweed, tomato, soybean, buckwheat, wheat gluten, oats, peanuts, rye, sugarcane and bread making yeast, which was level II or III before the treatment, and decreased to level I or II for kidney bean and string bean, which was levels IV to VI before the treatment. The eggs show no improvement due to not stopping the intake.

### Clinical Example 2

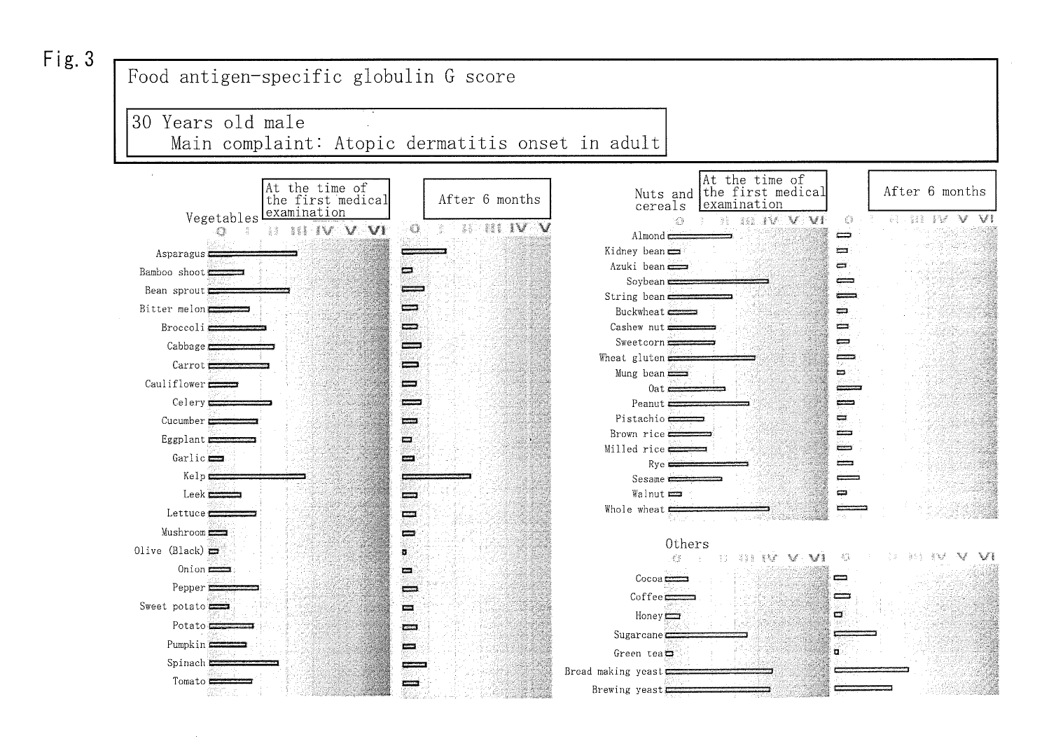

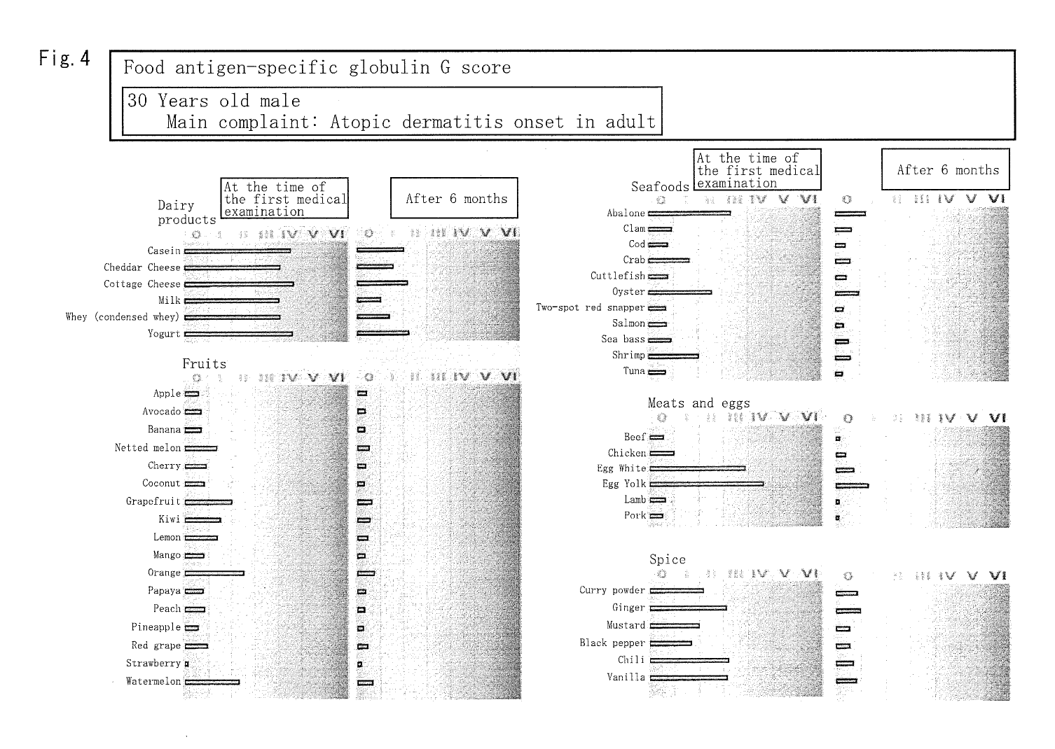

Clinical example 2 relates to atopic dermatitis developed in 30 years old male adult. FIGS. 3 and 4 show Ig G levels at the time of the first medical examination and after 6 month treatment for each food by the standard food panel. Similarly to Clinical Example 1, the subject was directed not to intake dairy products and eggs that showed level III or higher at the time of the first medical examination for 6 months.

A shown in FIG. 3, with respect to vegetables, the Ig G level was level III for any one of asparagus, bean sprouts and seaweed at the time of the first medical examination. However, after 6 months, the Ig G level became level II or lower. In addition, with respect to nuts and cereals, the Ig G level was level IV for soybean and whole wheat flour at the time of the first medical examination and level III for wheat gluten, peanuts and rye. However, the Ig G level decreased to nearly level 0 for any one of them. In addition, with respect to the other foods, the Ig G level was level III or IV for sugarcane, bread making yeast and brewing yeast at the time of the first medical examination, but decreased to levels I to III.

In addition, as shown in FIG. 4, with respect to the dairy products, the Ig G level was level IV for all, but decreased to level I or II. With respect to the fruits, the Ig G level was level I for netted melon, and level II for orange and watermelon, but became level 0 for all. With respect to the seafood, the Ig G level was level II or III for abalone, oyster and shrimp, but decreased to level 0 or I. With respect to the meats, the Ig G level was level III or IV for egg yolk and egg white, but became level 0 or I. With respect to the spices, the Ig G level was level III, but became level 0 for chili and vanilla.

The supplement of the invention contains 15 g of soybean peptide every dose. The fact that the Ig G for soybean decreases to level 0 from level IV with daily intake of the supplement of the invention supports that vulnerability of the intestinal environment and the intestinal tract barrier causes food-specific Ig G allergy. This result is contrary to the case of the eggs of Clinical example 1, and reflects that dairy products and eggs have a feature of easily forming Ig G allergy than soybean.

According to this invention, it is possible at least one of intestinal environment, intestinal tract barrier pertaining and immunological tolerance induction, as well as for improving IgG score of each food, and then this invention will be a contribution to improvement of following disease;
reflux esophagitis, inflammatory bowel diseases, irritable bowel syndrome, liver disfunction, premenstrual syndrome, migraine, chronic fatigue syndrome, hypoadrenaism (adrenal fatigue syndrome), autonomic dystonia (autonomic imbalance, or vegetative dystonia), menopausal syndrome, allergic diseases, asthma, atopic dermatitis, urticaria (haives),food allergy, pollinosis, inflammatory, psoriasis, autoimmune disorder, rheumatic disease, rheumatoid arthritis, rheumatic fever, SLE, etc, thyorid disease (Hashimoto's thyroiditis, morbus basedowii, thyroiditis, etc), infertility/autoimmune infertility, palmoplantar pustulosis.

What is claimed is:

1. A supplement for improving immunotolerance, intestinal environment and intestinal tract barrier which decreases an intestinal environment/barrier index, and which is obtained by mixing
an ingredient (A) and an ingredient (B),
the ingredient (A) comprise, at least any one of a probiotic, a biogenic and a prebiotic,
the probiotic is a microorganism giving beneficial effects to the host by improving balance of intestinal microorganism flora,
the biogenic is a food ingredient directly acting on a living body without intervention of intestinal flora,
the prebiotic is a substance selectively acting on useful bacteria living in the intestine, and promoting proliferation thereof or elevating activity thereof whereby to advantageously act on the health of the host, and
the ingredient (B) comprises at least one of L-glutamine, zinc, vitamin A and vitamin D,
the probiotic corresponds to any one of lactic acid bacteria, Lactobacillus bifidus, saccharified bacteria, natto bacteria, yeast fungus and others, or a mixture thereof, the biogenic corresponds to any one of casein phosphopeptide of a bioactive peptide, lactoferrin of multifunctional whey protein and docosahexaenoic acid or EPA (eicosapentaenoic acid) that is one kind of polyunsaturated fatty acid, or a mixture thereof, and the prebiotic corresponds to indigestible food ingredients such as oligosaccharide, dietary fiber, substance stimulating production of Lactobacillus bifidus, and others or a mixture thereof.

2. The supplement for improving immunotolerance, intestinal environment and intestinal tract barrier according to claim 1, wherein
the ingredient (A) comprises of L-glutamine, and
the ingredient (B) comprises at least one of zinc, vitamin A and vitamin D.

3. The supplement for improving immunotolerance, intestinal environment and intestinal tract barrier according to claim 1, wherein
the probiotic is spore lactic acid bacteria, and the prebiotic is indigestible food ingredients such as oligosaccharide, dietary fiber (polysaccharides), substance stimulating production of Lactobacillus bifidus and cyclic oligosaccharide.

4. The supplement for improving immunotolerance, intestinal environment and intestinal tract barrier according to claim 1, wherein
the zinc includes at least zinc among zinc and zinc gluconate, the vitamin A includes at least vitamin A among vitamin A, pre-vitamin A, carotenoid and vitamin A derivatives, and the vitamin D includes at least vitamin D among vitamin D, pre-vitamin D and vitamin D derivatives.

5. The supplement for improving immunotolerance, intestinal environment and intestinal tract barrier according to claim 1, wherein the supplement is obtained by mixing
an ingredient (C) comprising at least one of vitamin B group; calcium; magnesium; a magnesium compound; a functional nutritional ingredient comprising at least one of quercetin, lipopolysaccharide, lactoferrin and curcumin; protein; and amino acid with the ingredient (A) and the ingredient (B), and
the supplement decreases the intestinal environment/barrier index.

6. The supplement for improving immunotolerance, intestinal environment and intestinal tract barrier according to claim 4, wherein
the calcium includes shell calcium, the magnesium compound includes magnesium oxide, the protein includes soybean peptide and the amino acid includes L methionine.

7. The supplement for improving immunotolerance intestinal environment and intestinal tract barrier according to claim 1, wherein
when the ingredient (A) is spore lactic acid bacteria, the number of the spore lactic acid bacteria contained in recommended single dose is more than 25,000,000 bacteria number.

8. The supplement for improving immunotolerance, intestinal environment and intestinal tract barrier according to claim 1, wherein
when zinc is contained in the ingredient (B), the weight of zinc in recommended single dose is 10 to 120 mg.

9. The supplement for improving immunotolerance, intestinal environment and intestinal tract barrier according to claim 1, wherein
when L-glutamine is contained in the ingredient (B), the amount of L-glutamine contained in recommended single dose is 100 mg or more, and when L-glutamine is a main ingredient, the amount of L-glutamine is 1,000 to 5,000 mg.

10. The supplement for improving immunotolerance, intestinal environment and intestinal tract barrier according to claim 1, wherein
when vitamin A is a main ingredient of the ingredient (B), the amount of vitamin A contained in recommended single dose is 100 IU to 60,000 IU or corresponding amount of carotenoid.

11. The supplement for improving immunotolerance intestinal environment and intestinal tract barrier according to claim 1, wherein
when vitamin D is a main ingredient of the ingredient (B), the amount of vitamin D contained in recommended single dose is 100 IU to 10,000 IU or to 100,000 IU at intervals.

12. The supplement for improving immunotolerance, intestinal environment and intestinal tract barrier according to claim 1, wherein
the ingredient (A) comprises spore lactic acid bacteria as the probiotic, and oligosaccharide, cyclic oligosaccharide and inulin as the prebiotic, and the ingredient (B) includes zinc gluconate and vitamin A.

13. The supplement for improving immunotolerance, intestinal environment and intestinal tract barrier according to claim 12, wherein
the ingredient (C) comprises shell calcium as the calcium, magnesium oxide as the magnesium compound, soybean peptide as the protein, and L methionine as the amino acid.

14. The supplement for improving immunotolerance, intestinal environment and intestinal tract barrier according to claim 1, wherein
the supplement complements induction of immunotolerance such as allergen immunotherapy.
